SEQUENCE LISTING (3 sequences)

SEQ ID NO: 1 - protein, 13 aa, Artificial Sequence
  NCAPG2 pT1010 antigen
  MISC_FEATURE (7): Threonine is phosphorylated.
HRGVLSTLIAGPV

SEQ ID NO: 2 - protein, 1143 aa, Homo sapiens
  PEPTIDE (1)..(1143): Non-SMC condensin II complex, subunit G2 (NCAPG2)
MEKRETFVQAVSKELVGEFLQFVQLDKEASDPFSLNELLDELSRKQKEELWQRLKNLLTDVLLESPVDGWQVVEAQGEDNMETEHGSKMRKSIEIIYAITSVILASVSVINESENYEALLECVIILNGILYALPESERKLQSSIQDLCVTWWEKGLPAKEDTGKTAFVMLLRRSLETKTGADVCRLWRIHQALYCFDYDLEESGEIKDMLLECFININYIKKEEGRRFLSCLFNWNINFIKMIHGTIKNQLQGLQKSLMVYIAEIYFRAWKKASGKILEAIENDCIQDFMFHGIHLPRRSPVHSKVREVLSYFHHQKKVRQGVEEMLYRLYKPILWRGLKARNSEVRSNAALLFVEAFPIRDPNLHAIEMDSEIQKQFEELYSLLEDPYPMVRSTGILGVCKITSKYWEMMPPTILIDLLKKVTGELAFDTSSADVRCSVFKCLPMILDNKLSHPLLEQLLPALRYSLHDNSEKVRVAFVDMLLKIKAVRAAKFWKICPMEHILVRLETDSRPVSRRLVSLIFNSFLPVNQPEEVWCERCVTLVQMNHAAARRFYQYAHEHTACTNIAKLIHVIRHCLNACIQRAVREPPEDEEEEDGREKENVTVLDKTLSVNDVACMAGLLEIIVILWKSIDRSMENNKEAKLYTINKFASVLPEYLKVFKDDRCKIPLFMLMSFMPASAVPPFSCGVISTLRSREEGAVDKSYCTLLDCLCSWGQVGHILELVDNWLPTEHAQAKSNTASKGRVQIHDTRPVKPELALVYIEYLLTHPKNRECLLSAPRKKLNHLLKALETSKADLESLLQTPGGKPRGFSEAAAPRAFGLHCRLSIHLQHKFCSEGKVYLSMLEDTGFWLESKILSFIQDQEEDYLKLHRVIYQQIIQTYLTVCKDVVMVGLGDHQFQMQLLQRSLGIMQTVKGFFYVSLLLDILKEITGSSLIQKTDSDEEVAMLLDTVQKVFQKMLECIARSFRKQPEEGLRLLYSVQRPLHEFITAVQSRHTDTPVHRGVLSTLIAGPVVEISHQLRKVSDVEELTPPEHLSDLPPFSRCLIGIIIKSSNVVRSFLDELKACVASNDIEGIVCLTAAVHIILVINAGKHKSSKVREVAATVHRKLKTFMEITLEEDSIERFLYESSSRTLGELLNS

SEQ ID NO: 3 - protein, 14 aa, Artificial Sequence
  NCAPG2 pT1010 antigen
  MISC_FEATURE (11): Threonine is phosphorylated.
DTPVHRGVLSTLIA

(19) United States
(12) Patent Application Publication
(10) Pub. No.: US 2024/0279319 A1
(43) Pub. Date: Aug. 22, 2024

(54) ANTIBODY FOR IDENTIFYING PHOSPHORYLATION-SPECIFIC REACTIONS OF THREONINE AT POSITION 1010 OF NCAPG2, AND USE THEREOF

(71) Applicant: NATIONAL CANCER CENTER, Goyang-Si (KR)

(72) Inventors: Kyungtae KIM, Seoul (KR); Byung Il LEE, Goyang-si (KR); Joong-Won PARK, Seoul (KR); Eun Kyung HONG, Goyang-si (KR); Minji PARK, Paju-si (KR); Su-Hyung LEE, Gunpo-si (KR); Jin Sook KIM, Seoul (KR); Gyu Beom JANG, Goyang-si (KR); Nayoung HAN, Seoul (KR); Sung-Sik HAN, Seoul (KR); Sang Jae PARK, Goyang-si (KR); Sang Myung WOO, Seoul (KR)

(73) Assignee: NATIONAL CANCER CENTER, Goyang-Si (KR)

(21) Appl. No.: 18/564,010

(22) PCT Filed: May 24, 2022
(86) PCT No.: PCT/KR2022/007342

§ 371 Date: Nov. 24, 2023

(30) Foreign Application Priority Data
May 25, 2021 (KR) 10-2021-0066894

(51) Int. Cl. C07K 16/18; G01N 33/50; G01N 33/574

(52) U.S. Cl. CPC C07K 16/18; G01N 33/5011; G01N 33/57484; C07K 2317/34; G01N 2440/14

(57) ABSTRACT

The present invention relates to an antibody for identifying phosphospecific reactions of threonine at position 1010 in the amino acid sequence of non-SMC condensin II complex subunit G2 (NCAPG2), and a use thereof. It has been identified that: an antibody for identifying whether threonine at position 1010 in the amino acid sequence of NCAPG2 is phosphorylated, according to the present invention, has high selectivity and avidity with respect to pT1010 of NCAPG2; the reactivity thereof to pT1010 of NCAPG2 is inhibited in cancer cells treated with inhibitors of kinases for regulating cellular mitosis phase, such as CDK1, PLK1, Mps1, and aurora kinase; and detection of the antibody is far higher in tumor tissues than non-tumor tissues of a cancer patient through immunohistochemical staining. Therefore, it is expected that identifying the phosphorylation of threonine at position 1010 of NCAPG2 through the antibody, according to the present invention, can be effectively used in cancer-related research and development fields such as that of diagnosing cancer or screening for anticancer drug candidates.

This application claims priority to and the benefit of Korean Patent Application NO. 10-2021-0066894, filed on May 25, 2021, the disclosure of which is incorporated herein by reference in its entirety.

### TECHNICAL FIELD

The present invention relates to an antibody for confirming a phosphospecific reaction of threonine at position 1010 in the amino acid sequence of non-SMC condensin II complex subunit G2 (NCAPG2), and a use thereof.

### BACKGROUND ART

Cancer is one of the incurable diseases that mankind must solve, and huge capital is being invested worldwide in development to cure it. In Korea, cancer is the leading cause of death. Each year, more than 100,000 people are diagnosed with cancer, and more than 60,000 people die of cancer.

To date, most cancer detection methods are physical, including, for example, gastrointestinal X-ray imaging, such as double contrast, compression imaging, or mucosal imaging. Utilizing an endoscope allows for direct visual examination of internal organs, enabling not only the detection of even very small lesions that may not appear in an X-ray test but also a biopsy can be performed directly on sites suspected of cancer, thereby enhancing the accuracy of the diagnosis. However, this method comes with drawbacks such as hygiene concerns and the pain experienced by a patient during the test.

In addition, most cancer treatments that have been conducted so far are surgical resection of the lesion, and especially when the goal is complete cure, surgical removal is the only method. In such surgical removal, the principle is to resect as wide a range as possible in surgeries aimed at complete cure, but the range of removal may be determined by considering the aftereffects of extensive resection after surgery. However, even in this case, when cancer has spread to other organs, radical surgery is not possible. Therefore, at this time, other methods such as administration of an anticancer drug are chosen. Current anticancer drugs on the market provide only temporary effects such as symptom relief, reduced recurrence after removal, and extended lifespan. However, they have limitations in completely eradicating cancer and cause patients to doubly suffer due to side effects and economic burden, which are caused by the administration of anticancer drugs.

Accordingly, for cancer treatment, the development of a cancer diagnosis method with high sensitivity and specificity in a pre-treatment stage is most important, and such diagnosis must be possible in an early stage of cancer. Furthermore, personalized diagnostic and therapeutic approaches, including predicting a prognosis for a particular type of cancer, are required. However, to date, in cancer diagnosis, molecular diagnostic technology that specifically detects a lesion in an early stage and determines whether or not the disease has occurred is limited.

Meanwhile, mitosis refers to division in which all cell components are separated into two new cells. When mitosis begins, the condensation of chromosomes, the segregation and movement of mitotic spindles to opposite poles, the alignment of chromosomes in the middle, and finally the separation of all cell components occur. When cells begin to divide, chromosomes should form a specific structure for effective bidirectional segregation, such a mitosis-specific chromosomal structure mainly depends on three multiprotein complexes, two condensin complexes and one cohesin complex. The cohesin complex binds with its sister chromatid, and the condensin complex plays a role in making the inside of a chromosome thicker and shorter. Each condensin complex consists of an ATPase subunit heterodimer, i.e., chromosome structure-maintaining complex (structural maintenance of chromosomes, SMC 2 & SMC 4), and three non-SMC regulatory subunits. The unique sum of these three regulatory components defines each condensin complex. For example, NCAPD2, NCAPG, and NCAPH are components of condensin complex I, and NCAPD3, NCAPG2, and NCAPH2 are components of condensin complex II. The SMC 2-SMC 4 heterodimer is a crosslinker for mitotic DNA condensation using their ATPase activity. NCAPH and NCAPH2 are kleisin proteins that connect the SMC heterodimer with the other two regulatory subunits, and NCAPG, NCAPG2, NCAPD2, and NCAPD3 are regulatory subunits for each condensin complex, including a HEAT repeat domain corresponding to a variable scaffold. Condensin complex I is located in the cytosol during interphase, incorporated into chromosomes by aurora kinase B immediately after nuclear membrane collapse, and remains in the chromosome arm until cytokinesis. On the other hand, condensin complex II remains in the nucleus even during interphase and causes chromosome condensation during cell division, and the incorporation of condensin complex II into chromosomes is achieved through a catalytic activity-independent function of protein phosphatase 2A (PP2A). Several other actions including chromosomal decatenation, chromatin remodeling, and complex I condensation allow chromosome condensation to be maintained until cytokinesis. In addition, condensin complex I present in yeast species is a classical condensin complex for eukaryotic chromosome condensation. Condensin complex II regulates various cell actions such as chromosome rigidity, chromosome segregation, DNA repair, apoptosis, sister chromatid resolution, gene expression regulation, and histone modulation. Interestingly, homozygous mutants of all nematode condensin complex II components show abnormal sizes or heterogeneous nuclear distribution. In human cells, the deletion of any component of condensin complex II causes defects in chromosome alignment or division. In addition, NCAPD3 mainly affects centrosome division, and NCAPG2 deletion frequently causes chromosome misalignment in the metaphase plate. In relation to the chromosome division function, recent reports have shown that NCAPD3 contributes to the movement of PLK1 to the chromosome arm.

However, in the field of cancer-related research and development, such as cancer diagnosis and treatment, little is known about antibodies that specifically bind to a specific phosphorylated amino acid sequence in the amino acid sequence of NCAPG2 and the use thereof.

### DISCLOSURE

### Technical Problem

The present inventors confirmed that the phosphorylation of threonine at position 1010 in the amino acid sequence of non-SMC condensin II complex subunit G2 (NCAPG2) is associated with cancer, and prepared an antibody specific to NCAPG2 pT1010 to confirm the phosphorylation of threonine at position 1010 in the amino acid sequence of NCAPG2, and confirmed that the antibody shows high reactivity to NCAPG2 pT1010. Thus, the present invention was completed.

Accordingly, the present invention is directed to providing an antibody for confirming the phosphorylation of threonine at position 1010 in the amino acid sequence of NCAPG2, and a use thereof.

However, technical problems to be solved in the present invention are not limited to the above-described problems, and other problems, which are not described herein, will be fully understood by those of ordinary skill in the art from the following descriptions.

### Technical Solution

To achieve the above purpose, the present invention provides an antibody for confirming the phosphorylation of threonine at position 1010 from the N terminus of the amino acid sequence of non-SMC condensin II complex subunit G2 (NCAPG2).

Here, the antibody recognizes a peptide represented by the amino acid sequence of SEQ ID NO: 1 as an antigen, and specifically binds to threonine at position 1010 from the N terminus of the amino acid sequence of the phosphorylated NCAPG2.

In one embodiment of the present invention, the amino acid sequence of NCAPG2 may be represented by SEQ ID NO: 2.

In addition, the present invention provides a method of preparing an antibody for confirming the phosphorylation of threonine at position 1010 from the N terminus of the amino acid sequence of NCAPG2, which comprises isolating an antibody from serum isolated from an individual into which a peptide represented by the amino acid sequence of SEQ ID NO: 1 is injected.

In this method, the antibody has a relatively higher affinity for a peptide in which threonine, which is the 7^{th} amino acid from the N terminus of the peptide represented by the amino acid sequence of SEQ ID NO: 1, is phosphorylated, compared to a peptide in which threonine, which is the 7^{th} amino acid from the N terminus of the peptide represented by the amino acid sequence of SEQ ID NO: 1, is not phosphorylated.

In addition, the present invention provides a method of providing information for cancer diagnosis, which comprises confirming whether threonine at position 1010 from the N terminus of the amino acid sequence of NCAPG2 is phosphorylated by reacting cells or an animal, which expresses NCAPG2, with the antibody.

In one embodiment of the present invention, the method may further comprise diagnosing cancer when threonine at position 1010 from the N terminus of the amino acid sequence of NCAPG2 is phosphorylated.

In another embodiment of the present invention, the higher the degree of phosphorylation of the threonine at position 1010 from the N terminus of the amino acid sequence of NCAPG2, the lower the degree of differentiation of tumor cells.

In addition, the present invention provides a method of screening a candidate for an anticancer drug, which comprises: (a) treating cancer cells with a candidate;
(b) confirming whether threonine at position 1010 from the N terminus of the amino acid sequence of NCAPG2 is phosphorylated by reacting the cancer cells treated with the candidate with the antibody; and
(c) selecting the candidate as an anticancer drug candidate when the phosphorylation of threonine at position 1010 from the N terminus of the amino acid sequence of NCAPG2 in (b) is inhibited.

In one embodiment of the present invention, the anticancer drug candidate may comprise one or more selected from the group consisting of a polo-like kinase 1 (PLK1) inhibitor, a monopolar spindle 1 (Mps1) inhibitor, an aurora kinase inhibitor, and a cyclin-dependent kinase 1 (CDK1) inhibitor.

In another embodiment of the present invention, the PLK1 inhibitor, the Mps1 inhibitor, the aurora kinase inhibitor, or the CDK1 inhibitor may be one or more selected from the group consisting of a nucleotide, DNA, RNA, an amino acid, an aptamer, a protein, a compound, a natural substance, and a natural extract.

In addition, the present invention provides a kit for confirming the effectiveness of an anticancer drug candidate, which comprises:
the antibody; and
an anticancer drug candidate,
wherein the antibody serves as a prognostic indicator for confirming the effectiveness of the anticancer drug candidate.

In addition, the present invention provides a pharmaceutical composition for preventing or treating cancer, which comprises a material for inhibiting the phosphorylation of threonine at position 1010 from the N terminus of the amino acid sequence of NCAPG2.

In one embodiment of the present invention, the material may be one or more selected from the group consisting of a nucleotide, DNA, RNA, an amino acid, an aptamer, a protein, a compound, a natural substance, and a natural extract.

In addition, the present invention provides a method of diagnosing cancer, which comprises confirming whether threonine at position 1010 from the N terminus of the amino acid sequence of NCAPG2 is phosphorylated by reacting cells or an animal, which expresses NCAPG2, with the antibody.

In addition, the present invention provides a method of preventing or treating cancer, which comprises administering a material for inhibiting the phosphorylation of threonine at position 1010 from the N terminus of the amino acid sequence of NCAPG2 to an individual in need thereof.

In addition, the present invention provides a use of a material for inhibiting the phosphorylation of threonine at position 1010 from the N terminus of the amino acid sequence of NCAPG2 for preventing or treating cancer.

In addition, the present invention provides a use of a material for inhibiting the phosphorylation of threonine at position 1010 from the N terminus of the amino acid sequence of NCAPG2 for preparing a drug for preventing or treating cancer.

### Advantageous Effects

According to the present invention, it was confirmed that an antibody for confirming whether threonine at position 1010 in the amino acid sequence of non-SMC condensin II complex subunit G2 (NCAPG2) is phosphorylated has high selectivity and high binding ability to NCAPG2 pT1010, inhibits the reactivity to NCAPG2 pT1010 in cancer cells treated with an inhibitor against phosphorylases for regulating mitosis, such as CDK1, PLK1, Mps1, and aurora kinase, and is detected at a very high level in tumor tissue, compared to non-tumor tissue of a cancer patient, through immunohistochemical staining. Therefore, by confirming the phosphorylation of threonine at position 1010 of NCAPG2 using the antibody according to the present invention, it is expected to be useful for cancer-related research and development fields, including cancer diagnosis or screening of an anticancer drug candidate.

### DESCRIPTION OF DRAWINGS

FIG. 1A is a view that confirms the reactivity of an antibody against a C-HRGVLS(pT)LIAGPV-amide antigen, compared to a reaction with a variant of non-SMC condensin II complex subunit G2 (NCAPG2) in which threonine at position 1010 is substituted with alanine according to one embodiment of the present invention.

FIGS. 1B and 1C are views that confirm the reactivity of an anti-pT1010 antibody against the C-HRGVLS(pT)LIAGPV-amide antigen in cells treated with a phosphorylase inhibitor according to one embodiment of the present invention.

FIG. 2A is a view showing an immunohistochemical staining result using an anti-NCAPG2 antibody when NCAPG2 expression is reduced according to one embodiment of the present invention.

FIG. 2B is a view showing an immunohistochemical staining result using an anti-pT1010 antibody against a CDTPVHRGVLSpTLIA antigen when NCAPG2 expression is reduced according to one embodiment of the present invention.

FIG. 2C is a view showing an immunohistochemical staining result using an anti-pT1010 antibody against the C-HRGVLS(pT)LIAGPV-amide antigen when NCAPG2 expression is reduced according to one embodiment of the present invention.

FIG. 3 is a view showing an immunohistochemical result when normal cells and cancer cells are treated with an anti-pT1010 antibody against the C-HRGVLS(pT)LIAGPV-amide antigen according to one embodiment of the present invention.

FIG. 4A is a view showing an immunohistochemical result in liver cancer tissue for confirming the expression of NCAPG2 pT1010 according to one embodiment of the present invention.

FIG. 4B is a view showing a quantified result using the H-score system for a positive reaction after immunohistochemical staining in liver cancer tissue for confirming the expression of NCAPG2 pT1010 according to one embodiment of the present invention.

FIG. 4C is a view showing an immunohistochemical result in cholangiocarcinoma tissue for confirming the expression of NCAPG2 pT1010 according to one embodiment of the present invention.

FIG. 4D is a view showing an immunohistochemical result in pancreatic cancer tissue for confirming the expression of NCAPG2 pT1010 according to one embodiment of the present invention.

FIG. 5A is a view confirming NCAPG2 pT1010 expression according to the degree of tumor differentiation in pancreatic cancer tissue according to one embodiment of the present invention through immunohistochemistry.

FIG. 5B is a view confirming NCAPG2 pT1010 expression according to the degree of tumor differentiation in pancreatic cancer tissue according to one embodiment of the present invention using the Q-score system.

### BEST MODE

The present invention provides an antibody for confirming whether threonine at position 1010 from the N terminus of the amino acid sequence of non-SMC condensin II complex subunit G2 (NCAPG2) is phosphorylated.

Here, the antibody recognizes a peptide represented by the amino acid sequence of SEQ ID NO: 1 as an antigen, and specifically binds to threonine at position 1010 from the N terminus of the amino acid sequence of the phosphorylated NCAPG2.

The NCAPG2 may be derived from a human, and may be represented by the amino acid sequence of SEQ ID NO: 2 (NCBI GenBank: AAH43404.1).

In the present invention, “threonine at position 1010 of NCAPG2” or “threonine at position 1010 in the amino acid sequence of NCAPG2” may refer to threonine at position 1010 from the N terminus of the amino acid sequence of NCAPG2.

The amino acid sequence of NCAPG2 represented by SEQ ID NO: 2 comprises threonine, which is the amino acid at position 1010 from the N terminus of the amino acid sequence, and “pT1010” used herein refers to threonine, which is the amino acid at position 1010 from the N terminus of the amino acid sequence of NCAPG2, to which a phosphate group binds.

In addition, the present invention provides a method of preparing an antibody for confirming the phosphorylation of threonine at position 1010 from the N terminus of the amino acid sequence of NCAPG2, which comprises isolating an antibody from serum isolated from an individual into which a peptide represented by the amino acid sequence of SEQ ID NO: 1 is injected,
wherein the antibody has a relatively higher affinity for a peptide in which threonine, which is the 7^{th} amino acid from the N terminus of the peptide represented by the amino acid sequence of SEQ ID NO: 1, is phosphorylated, compared to a peptide in which threonine, which is the 7^{th} amino acid from the N terminus of the peptide represented by the amino acid sequence of SEQ ID NO: 1, is not phosphorylated.

The term “phosphorylation” used herein is a biochemical reaction in which a phosphate group is added to a serine (S), threonine (T) or tyrosine (Y) residue of a specific protein, and is catalyzed by a protein kinase. Phosphorylation usually regulates protein activity by modifying the function of a target protein. As a part of a cell homeostasis mechanism, phosphorylation is just a temporary process and is reversed by other enzymes called phosphatases. Any abnormality in either side of the reaction (phosphorylation vs. dephosphorylation) may disrupt cell function.

The term “antibody” used herein refers to a polypeptide including a framework region derived from an immunoglobulin gene or a fragment thereof, which specifically binds to and recognizes an antigen. The immunoglobulin gene includes numerous immunoglobulin variable region genes, including κ, λ, α, γ, δ, ε, and η constant region genes. Light chains are classified as κ or λ. Heavy chains are classified as γ, η, α, δ, or ε, designating the immunoglobulin class IgG, IgM, IgA, IgD, or IgE, respectively. Typically, the antigen-binding region of an antibody plays a key role in binding specificity and affinity. In the present invention, an antibody or a fragment thereof may be derived from different organisms, including a human, a mouse, a rat, a hamster, a camel, and a rabbit, and according to one embodiment of the present invention, derived from a rabbit, but the present invention is not limited thereto. The antibody of the present invention may include an antibody that is modified or mutated at one or more amino acid sites to improve or adjust a preferable function (e.g., glycosylation, expression, antigen recognition, effector function, antigen binding, specificity, etc.) of the antibody.

In the present invention, the antibody may be prepared by injecting a C-HRGVLS(pT)LIAGPV-amide peptide represented by the amino acid sequence of SEQ ID NO: 1 into an individual to be recognized as an antigen. Here, the antibody has the highest specificity for pT1010. The peptide may be conjugated with keyhole limpet hemocyanin (KLH) before injection into an individual, but the present invention is not limited thereto. In addition, the peptide may be modified at the N and C termini to improve similarity to a natural protein and safety. According to one embodiment of the present invention, in the peptide represented by the amino acid sequence of SEQ ID NO: 1, the N terminus is acetylated and the C terminus is amidated, but the present invention is not limited thereto.

In the present invention, the binding site and type of the antibody are not limited as long as the antibody can confirm phosphorylation in threonine at position 1010 from the N terminus of the amino acid sequence of NCAPG2. For example, the antibody may specifically bind to a specific amino acid sequence of NCAPG2, and specifically bind to the phosphate group of threonine at position 1010 from the N terminus of the amino acid sequence of NCAPG2.

The term “specifically binding” refers to antibody binding to a certain antigen, and an antibody binds to the certain antigen with an affinity higher, i.e., at least two times higher than that for binding to an unrelated antigen (e.g., BSA or casein) other than the certain antigen or a closely related antigen.

In addition, the present invention provides a method of providing information for cancer diagnosis, which comprises confirming whether threonine at position 1010 from the N terminus of the amino acid sequence of NCAPG2 is phosphorylated by reacting NCAPG2-expressing cells with the antibody.

In addition, the present invention provides a method of diagnosing cancer, which comprises confirming whether threonine at position 1010 from the N terminus of the amino acid sequence of NCAPG2 is phosphorylated by reacting cells or an animal, which expresses NCAPG2, with the antibody.

In the present invention, the method may further comprise diagnosing cancer when threonine at position 1010 from the N terminus of the amino acid sequence of NCAPG2 is phosphorylated, and the higher the degree of phosphorylation of threonine at position 1010 from the N terminus of the amino acid sequence of NCAPG2, the lower the degree of differentiation of tumor cells.

The term “diagnosis” used herein refers to confirming the presence or characteristics of a pathological condition. For the purpose of the present invention, diagnosis is the determination of the occurrence of cancer.

In addition, the present invention provides a method of treating cancer, which comprises the following steps:
(a) confirming whether threonine at position 1010 from the N terminus of the amino acid sequence of NCAPG2 is phosphorylated by reacting cells or an animal, which expresses NCAPG2, with the antibody;
(b) diagnosing cancer when threonine at position 1010 from the N terminus of the amino acid sequence of NCAPG2 is phosphorylated; and
(c) treating the cancer diagnosed in (b).

In the present invention, in (c), for treating cancer, a method such as chemotherapy, radiotherapy, surgery, or a biological therapy may be used.

In the present invention, the chemotherapy refers to the act of using chemicals to treat a specific disease and all drugs used at that time.

In the present invention, the drug may be one or more anticancer drugs selected from the group consisting of, for example, paclitaxel, doxorubicin, 5-fluorouracil, cisplatin, imatinib, carboplatin, oxaliplatin, tegafur, irinotecan, docetaxel, cyclophosphamide, cemcitabine, ifosfamide, mitomycin C, vincristine, etoposide, methotrexate, topotecan, tamoxifen, vinorelbine, camptothecin, danuorubicin, chlorambucil, bryostatin-1, calicheamicin, mayatansine, levamisole, DNA recombinant interferon alfa-2a, mitoxantrone, nimustine, interferon alfa-2a, doxifluridine, formestane, leuprolide acetate, megestrol acetate, carmofur, teniposide, bleomycin, carmustine, heptaplatin, exemestane, anastrozole, estramustine, capecitabine, goserelin acetate, polysaccharide potassium, medroxypogesterone acetate, epirubicin, letrozole, pirarubicin, topotecan, altretamine, toremifene citrate, BCNU, taxotere, actinomycin D, synthetic analogues thereof, and materials modified or exhibiting the same efficacy, but the present invention is not limited thereto.

In the present invention, the radiotherapy refers to irradiating a patient with high-energy radiation, including, but is not limited to, x-rays, gamma rays, and neutrons. This type of therapy may include, but is not limited to, external beam therapy, internal radiation therapy, implant radiation, brachytherapy, and systemic radiation therapy, but the present invention is not limited thereto.

In the present invention, “surgery” includes any therapeutic or diagnostic procedure involving the methodical action of hands or hands together with a device with respect to the body of an individual to achieve a curative, therapeutic or diagnostic effect.

In the present invention, “biological therapy” means a therapeutic method that directly/indirectly uses the immune system of the human body by using a biological agent containing a material derived from an organism or an organism, and the biological agent includes vaccines whose potency and safety cannot be evaluated by physical or chemical tests alone, allergens, antigens, hormones, cytokines, enzymes, blood and plasma, immune sera, monoclonal antibodies, fermentation products, antitoxins, and laboratory diagnostics.

In the present invention, the biological agent may be one or more selected from the group consisting of, for example, adalimumab, alemtuzumab, bevacizumab, cetuximab, daratumumab, panitumumab, rituximab, trastuzumab, pertuzumab, ipilimumab, nivolumab, pembrolizumab, atezolizumab, durvalumab, avelumab, tocilizumab, sarilumab, satralizumab, and siltuximab, but the present invention is not limited thereto.

In the present invention, the types of “cells” may include cells derived from vertebrates, such as mammals including humans (humans, monkeys, mice, rats, hamsters, cows, etc.), birds (chickens, ostriches, etc.), amphibians (frogs, etc.), and fish, or invertebrates, such as insects (silkworms, moths, fruit flies, etc.), plants, microorganisms such as yeast, and preferably cells derived from a mammal including a human, but the present invention is not limited thereto.

The term “cancer” used herein encompasses diseases caused by cells with aggressive characteristics in which cells divide and grow while disregarding normal growth limits, invasive characteristics in which cells infiltrate into surrounding tissue, and metastatic characteristics in which cells spread to other parts of the body.

In the present invention, the cancer may be any type of cancer known as a malignant tumor in the art without any particular limitation, and may be selected from, for example, breast cancer, colorectal cancer, lung cancer, small cell lung cancer, stomach cancer, liver cancer, blood cancer, bone cancer, cholangiocarcinoma, pancreatic cancer, skin cancer, head or neck cancer, dermal or intraocular melanoma, uterine cancer, ovarian cancer, rectal cancer, anal cancer, colon cancer, fallopian tube carcinoma, endometrial carcinoma, cervical cancer, vaginal cancer, vulvar carcinoma, Hodgkin's disease, esophageal cancer, small intestine cancer, endocrine adenocarcinoma, thyroid cancer, parathyroid cancer, adrenal cancer, soft tissue sarcoma, urethral cancer, penile cancer, prostate cancer, chronic or acute leukemia, lymphocytic lymphoma, bladder cancer, kidney or ureter cancer, renal cell carcinoma, renal pelvis carcinoma, CNS tumor, primary CNS lymphoma, spinal cord tumors, brainstem gliomas, and pituitary adenomas, and according to one embodiment of the present invention, the cancer is preferably breast cancer, liver cancer, cholangiocarcinoma or pancreatic cancer.

In the present invention, “reacting cells or an animal with an antibody” refers to treating cells or an animal with the antibody or bringing the antibody into contact with cells or an animal to confirm whether threonine at position 1010 from the N terminus of the amino acid sequence of NCAPG2 is phosphorylated.

In the present invention, “contact” has its normal meaning, and means combining two or more agents (e.g., two polypeptides) or combining an agent with cells (e.g., a protein and cells). Contact may occur in vitro. For example, contact means combining two or more agents in a test tube or different containers or combining a cell lysate with a test agent. In addition, contact may occur in cells or in situ. For example, two polypeptides are brought into contact in cells or a cell lysate by co-expressing recombinant polynucleotides encoding two polypeptides in cells.

In addition, the present invention provides a method of screening an anticancer drug candidate, including:
(a) treating cancer cells with a candidate;
(b) confirming whether threonine at position 1010 from the N terminus of the amino acid sequence of NCAPG2 is phosphorylated by reacting the cancer cells treated with the candidate with the antibody; and
(c) selecting the candidate as an anticancer drug candidate when the phosphorylation of threonine at position 1010 from the N terminus of the amino acid sequence of NCAPG2 in (b) is inhibited.

In addition, the present invention provides a kit for confirming the efficacy of an anticancer drug candidate, including:
the antibody; and
an anticancer drug candidate,
wherein the antibody serves as a prognostic indicator for confirming the effectiveness of the anticancer drug candidate.

The term “candidate” used herein refers to an unknown material used in screening to confirm whether threonine at position 1010 from the N terminus of the amino acid sequence of NCAPG2 is phosphorylated, and according to one embodiment of the present invention, a candidate for inhibiting the phosphorylation of threonine at position 1010 from the N-terminus of the amino acid sequence of NCAPG2 may be selected as an anticancer drug candidate, and the anticancer drug candidate may be one or more selected from the group consisting of a polo-like kinase 1 (PLK1) inhibitor, a monopolar spindle 1 (Mps1) inhibitor, an aurora kinase inhibitor, and a cyclin-dependent kinase 1 (CDK1) inhibitor. In the present invention, the PLK1 inhibitor, Mps1 inhibitor, aurora kinase inhibitor, or CDK1 inhibitor may be one or more selected from the group consisting of a nucleotide, DNA, RNA, an amino acid, an aptamer, a protein, a compound, a natural substance, and a natural extract. However, any material that can inhibit PLK1, Mps1, aurora kinase, or CDK1 is used without limitation.

In the present invention, the prognostic indicator may provide some information on a tumor size, a lymph node condition, a histological grade as well as a prognosis, and present the possibility of a response to a specific therapeutic agent, and may be used for selecting a patient for treatment with a specific therapeutic agent. According to one embodiment of the present invention, an antibody specific to NCAPG2 pT1010 may serve as a prognostic indicator for confirming the potential of the PLK1 inhibitor, Mps1 inhibitor, aurora kinase inhibitor, or CDK1 inhibitor as an anticancer drug by confirming whether the phosphorylation of threonine at position 1010 of NCAPG2 is inhibited by the treatment with a PLK1 inhibitor, Mps1 inhibitor, aurora kinase inhibitor, or CDK1 inhibitor.

In the present invention, the kit may confirm the efficacy of an anticancer drug candidate, and to confirm the efficacy of the anticancer drug candidate, the kit may comprise an antibody specifically binding to threonine at position 1010 from the N terminus of the amino acid sequence of NCAPG2 and one or more anticancer drug candidates selected from the group consisting of a PLK1 inhibitor, an Mps1 inhibitor, an aurora kinase inhibitor, and a CDK1 inhibitor, and the antibody and the anticancer drug candidate(s) may be applied one or more times without limitation. In addition, there is no limit on the order of application of the antibody and anticancer drug candidate(s), and their application may occur simultaneously or at different times.

In the present invention, the kit may comprise a container; instructions; an antibody specifically binding to threonine at position 1010 from the N terminus of the amino acid sequence of NCAPG2; and one or more anticancer drug candidates selected from the group consisting of a PLK1 inhibitor, an Mps1 inhibitor, an aurora kinase inhibitor, and a CDK1 inhibitor. The container may serve to package the antibody specifically binding to threonine at position 1010 in the amino acid sequence of NCAPG2, and one or more anticancer drug candidates selected from a PLK1 inhibitor, an Mps1 inhibitor, an aurora kinase inhibitor, and a CDK1 inhibitor, respectively, and store and fix them. A material for the container may be, for example, a plastic or glass bottle, but the present invention is not limited thereto. In addition, the instructions included in the kit for confirming the efficacy of an anticancer drug candidate according to the present invention may contain protocols for confirming the efficacy of the anticancer drug candidate. The protocols may be described on a sheet or booklet separate from the container, and the sheet or booklet may be included with an antibody specifically binding to threonine at position 1010 from the N terminus of the amino acid sequence of NCAPG2, and one or more anticancer drug candidates selected from the group consisting of a PLK1 inhibitor, an Mps1 inhibitor, an aurora kinase inhibitor, and a CDK1 inhibitor in the container.

In the present invention, a method of confirming whether threonine at position 1010 from the N terminus of the amino acid sequence of NCAPG2 is phosphorylated using an antibody specific to NCAPG2 pT1010 is a conventional method known in the art, including one or more methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony immunodiffusion, complement fixation assay, a protein chip, immunocytochemistry, and immunohistochemistry, and according to an preferable embodiment of the present invention, the method may be immunoprecipitation, immunocytochemistry, or immunohistochemistry, but the present invention is not limited thereto.

In addition, the present invention provides a pharmaceutical composition for preventing or treating cancer, which comprises a material for inhibiting the phosphorylation of threonine at position 1010 from the N terminus of the amino acid sequence of NCAPG2.

In addition, the present invention provides a method of preventing or treating cancer, which comprises administering a material for inhibiting the phosphorylation of threonine at position 1010 from the N terminus of the amino acid sequence of NCAPG2 to an individual in need thereof.

In addition, the present invention provides a use of a material for inhibiting the phosphorylation of threonine at position 1010 from the N terminus of the amino acid sequence of NCAPG2 for preventing or treating cancer.

In addition, the present invention provides a use of a material for inhibiting the phosphorylation of threonine at position 1010 from the N terminus of the amino acid sequence of NCAPG2 for preparing a drug for preventing or treating cancer.

In the present invention, the material for inhibiting the phosphorylation of threonine at position 1010 from the N terminus of the amino acid sequence of NCAPG2 may comprise one or more selected from the group consisting of a nucleotide, DNA, RNA, an amino acid, an aptamer, a protein, a compound, a natural substance, and a natural extract, but the present invention is not limited thereto.

In the present invention, “prevention” refers to all actions suppressing cancer or delaying the onset of cancer by administering the composition according to the present invention.

In the present invention, “treatment” refers to all actions involved in alleviating or beneficially changing symptoms of cancer by administering the composition according to the present invention.

In the present invention, “administration” refers to providing the composition of the present invention to an individual in any appropriate manner.

In the present invention, “individual” refers to a target to which the composition of the present invention is administered, and more specifically, includes a human or a non-human primate, and mammals such as mice, rabbits, dogs, cats, horses, and cattle.

The pharmaceutical composition according to the present invention may further include appropriate carriers, excipients, and diluents, usually used in the preparation of the pharmaceutical composition. The excipients may include, for example, one or more selected from the group consisting of a diluent, a binder, a disintegrant, a lubricant, an adsorbent, a humectant, a film-coating material, and a controlled-release additive.

The dosage form of the pharmaceutical composition according to the present invention may be a powder, a granule, a sustained-release granule, an enteric granule, a liquid, an ophthalmic solution, an elixir, an emulsion, a suspension, a spirit, a troche, aromatic water, a lemonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a perfusate, a plaster, a lotion, a paste, a spray, an inhalant, a patch, a sterile injection, or an external preparation such as an aerosol according to a conventional method, and the external preparation may be formulated as a cream, a gel, a patch, a spray, an ointment, a plaster, a lotion, a liniment, a paste, or a cataplasma according to a conventional method.

The carrier, excipient, and diluent, which may be included in the pharmaceutical composition according to the present invention, may include lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil.

The pharmaceutical composition according to the present invention is formulated with diluents or excipients, such as a filler, a thickening agent, a binder, a wetting agent, a disintegrant, and a surfactant, which are commonly used.

As additives for a tablet, powder, granule, capsule, pill and troche, excipients such as corn starch, potato starch, wheat starch, lactose, sucrose, glucose, fructose, di-mannitol, precipitated calcium carbonate, synthetic aluminum silicate, calcium monohydrogen phosphate, calcium sulfate, sodium chloride, sodium bicarbonate, purified lanolin, microcrystalline cellulose, dextrin, sodium alginate, methylcellulose, carboxymethylcellulose, sodium carboxymethylcellulose, kaolin, urea, colloidal silica gel, hydroxypropyl starch, hydroxypropyl methyl cellulose, 1928, 2208, 2906, 2910, propylene glycol, casein, calcium lactate and Primojel; binders such as gelatin, gum arabic, ethanol, agar powder, cellulose acetate phthalate, carboxymethyl cellulose, carboxymethyl cellulose calcium, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch powder, hydroxypropylcellulose, hydroxypropylmethylcellulose, polyvinyl alcohol and polyvinylpyrrolidone; disintegrants such as hydroxypropylmethylcellulose, corn starch, agar powder, methylcellulose, bentonite, hydroxypropyl starch, sodium carboxymethylcellulose, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, gum arabic, amylopectin, pectin, sodium polyphosphate, ethyl cellulose, sucrose, magnesium aluminum silicate, a di-sorbitol solution and light anhydrous silicic acid; and lubricants such as calcium stearate, magnesium stearate, stearic acid, hydrogenated vegetable oil, talc, limestone kaolin, petrolatum, sodium stearate, cacao butter, sodium salicylate, magnesium salicylate, polyethylene glycol 4000 and, 6000, liquid paraffin, hydrogenated soy bean oil (Lubri wax), aluminum stearate, zinc stearate, sodium lauryl sulfate, magnesium oxide, Macrogol, synthetic aluminum silicate, silicic anhydride, a higher fatty acid, a higher alcohol, silicone oil, paraffin oil, polyethylene glycol fatty acid ether, starch, sodium chloride, sodium acetate, sodium oleate, dileucine and light anhydrous silicic acid, may be used.

Additives for a liquid according to the present invention may be water, diluted hydrochloric acid, diluted sulfuric acid, sodium citrate, monostearate sucrose, polyoxyethylene sorbitol fatty acid esters (Tween esters), polyoxyethylene monoalkylethers, lanolin ethers, lanolin esters, acetic acid, hydrochloric acid, acetic acid, hydrochloric acid, aqueous ammonia, ammonium carbonate, potassium hydroxide, sodium hydroxide, prolamine, polyvinylpyrrolidone, ethyl cellulose, and sodium carboxymethylcellulose.

For a syrup according to the present invention, white sugar, other types of sugar, or sweeteners, and if necessary, a flavoring agent, a colorant, a preservative, a stabilizer, a suspending agent, an emulsifier, or a thickening agent may be used.

For an emulsion according to the present invention, distilled water may be used, and if necessary, an emulsifier, a preservative, a stabilizer, or a fragrance may be used.

For a suspension according to the present invention, a suspending agent such as acacia, tragacanth, methylcellulose, carboxymethylcellulose, sodium carboxymethylcellulose, microcrystalline cellulose, sodium alginate, hydroxypropylmethylcellulose (HPMC), HPMC 1828, HPMC 2906, or HPMC 2910 may be used, and if necessary, a surfactant, a preservative, a stabilizer, a colorant, and a fragrance may be used.

For an injection according to the present invention, a solvent such as injectable sterile water, 0.9% sodium chloride for injection, Ringer's solution, a dextrose for injection, dextrose+sodium chloride for injection, PEG, lactated Ringer's solution, ethanol, propylene glycol, non-volatile oil-sesame oil, cottonseed oil, peanut oil, soybean oil, corn oil, ethyl oleate, isopropyl myristic acid or benzene benzoate; a solubilizing agent such as sodium benzoate, sodium salicylate, sodium acetate, urea, urethane, monoethylacetamine, butazolidine, propylene glycol, Tween, nicotinamide, hexamine or dimethylacetamide; a buffer such as a weak acid and a salt thereof (acetic acid and sodium acetate), a weak base and a salt thereof (ammonia and ammonium acetate), an organic compound, a protein, albumin, peptone, or gums; an isotonic agent such as sodium chloride; a stabilizer such as sodium bisulfite (NaHSO₃), carbon dioxide gas, sodium metabisulfite (Na₂S₂O₃), sodium sulfite (Na₂SO₃), nitrogen gas (N₂) or ethylenediaminetetracetic acid; an antioxidant such as sodium bisulfide 0.1%, sodium formaldehyde sulfoxylate, thiourea, disodium ethylenediaminetetraacetate or acetone sodium bisulfite; a pain-relief agent such as benzyl alcohol, chlorobutanol, procaine hydrochloride, glucose or calcium gluconate; or a suspending agent such as sodium CMC, sodium alginate, Tween 80 or aluminum monostearate, may be used.

For a suppository according to the present invention, a base such as cacao butter, lanolin, Witepsol, polyethylene glycol, glycerogelatin, methyl cellulose, carboxymethylcellulose, a mixture of stearate and oleate, Subanal, cottonseed oil, peanut oil, palm oil, cacao butter+cholesterol, lecithin, Lanette wax, glycerol monostearate, Tween or Span, Imhausen, monolene (propylene glycol monostearate), glycerin, Adeps solidus, Buytyrum Tego-G, Cebes Pharma 16, hexalide base 95, Cotomar, Hydrokote SP, S-70-XXA, S-70-XX75 (S-70-XX95), Hydrokote 25, Hydrokote 711, Idropostal, Massa estrarium, A, AS, B, C, D, E, I, T), Mass-MF, Masupol, Masupol-15, neosuppostal-N, paramount-B, supposiro (OSI, OSIX, A, B, C, D, H, L), suppository base IV types (AB, B, A, BC, BBG, E, BGF, C, D, 299), Suppostal (N, Es), Wecoby (W, R, S, M, Fs), or a Tegester triglyeride base (TG-95, MA, 57) may be used.

A solid formulation for oral administration may be a tablet, a pill, a powder, a granule or a capsule, and such a solid formulation may be prepared by mixing at least one of excipients, for example, starch, calcium carbonate, sucrose, lactose and gelatin, with the active ingredient. Also, in addition to the simple excipient, lubricants such as magnesium stearate and talc may also be used.

As a liquid formulation for oral administration, a suspension, a liquid for internal use, an emulsion, or a syrup may be used, and a generally-used simple diluent such as water or liquid paraffin, as well as various types of excipients, for example, a wetting agent, a sweetener, a fragrance and a preservative may be included. A formulation for parenteral administration may be a sterilized aqueous solution, a non-aqueous solvent, a suspension, an emulsion, a lyophilizing agent or a suppository. As the non-aqueous solvent or suspension, propylene glycol, polyethylene glycol, a vegetable oil such as olive oil, or an injectable ester such as ethyl oleate may be used.

The pharmaceutical composition according to the present invention is administered at a pharmaceutically effective amount. The “pharmaceutically effective amount” used herein refers to an amount sufficient for treating a disease at a reasonable benefit/risk ratio applicable for medical treatment, and an effective dosage may be determined by parameters including the type and severity of a patient's disease, drug activity, sensitivity to a drug, administration time, an administration route and an excretion rate, the duration of treatment and drugs simultaneously used, and other parameters well known in the medical field.

The pharmaceutical composition according to the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without side effects, and such a dose may be easily determined by one of ordinary skill in the art.

The pharmaceutical composition of the present invention may be administered into a subject via various routes. All administration routes may be expected, and the pharmaceutical composition of the present invention may be administered by, for example, oral administration, subcutaneous injection, intraperitoneal administration, intravenous, intramuscular or intrathecal injection, sublingual administration, buccal administration, rectal insertion, vaginal insertion, ocular administration, ear administration, nasal administration, inhalation, spraying through the mouth or nose, skin administration, or transdermal administration.

The pharmaceutical composition of the present invention is determined depending on the type of drug as the active ingredient along with various related factors such as a disease to be treated, the route of administration, a patient's age, sex, and weight, and the severity of a disease.

In one embodiment of the present invention, an antibody for confirming the phosphorylation (pT1010) of threonine at position 1010 from the N terminus in the amino acid sequence of NCAPG2 was prepared using C-HRGVLS(pT)LIAGPV-amide as an antigen (see Example 1).

In another embodiment of the present invention, by comparing the antibody reactivity of a variant (TA) in which a threonine (Thr) prototype at position 1010 from the N terminal in the amino acid sequence of NCAPG2 was substituted with alanine (Ala) with an antibody against a CDTPVHRGVLSpTLIA antigen, it was confirmed that the prepared antibody has much higher specificity for NCAPG2 pT1010, and it was confirmed by immunoprecipitation that a band specific to NCAPG2 pT1010 was reduced by the treatment with a phosphorylase inhibitor (see Example 2).

In still another embodiment of the present invention, the specificity of the prepared antibody was confirmed by immunocytochemistry, and it was confirmed that, when NCAPG2-specific expression was reduced using siRNA, the reactivity of the anti-pT1010 antibody against the C-HRGVLS(pT)LIAGPV-amide antigen has sufficient specificity, and it can be seen that the selectivity or binding degree of the antibody is more suitable than an antibody against the CDTPVHRGVLSpTLIA antigen (see Example 3).

In still another embodiment of the present invention, as a result of comparing immunostaining patterns in normal cells and cancer cells, it was confirmed that, when the normal cells were treated with an anti-pT1010 antibody, kinetochores was specifically stained during the cell division phase, but in the cancer cells, the staining became more intense, and in many cases, kinetochore-specific staining did not occur during the cell division phase (see Example 4).

In yet another embodiment of the present invention, immunohistochemistry was performed using the prepared antibody to confirm the expression of NCAPG2 pT1010 in non-tumor areas and tumor areas of liver cancer tissue, cholangiocarcinoma tissue, and pancreatic cancer tissue, and it was confirmed that NCAPG2 pT1010 was expressed in the nuclei of most cells and its expression in the tumor areas was significantly higher compared to the non-tumor areas (see Example 5).

In yet another embodiment of the present invention, as a result of immunohistochemistry performed on NCAPG2 pT1010 in pancreatic cancer tissue, it was confirmed that, the degree of NCAPG2 pT1010 expression is associated with the degree of tumor differentiation in pancreatic cancer tissue (see Example 6).

### MODE FOR INVENTION

Hereinafter, preferred examples are presented to better understand the present invention. However, the following examples are provided to more easily understand the present invention, and the content of the present invention is not limited by the following examples.

### Example 1. Preparation of NCAPG2 pT1010-Specific Antibody

To prepare an antibody to confirm the phosphorylation of threonine at position 1010 (pT1010) in the amino acid sequence of non-SMC condensin II complex subunit G2 (NCAPG2), a peptide with a purity of more than 90% was synthesized using C-HRGVLS(pT)LIAGPV-amide (SEQ ID NO: 1) as an antigen. Here, after conjugation with keyhole limpet hemocyanin (KLH), the resulting peptide was injected into a rabbit. The peptide was repeatedly injected 4 or 5 times to produce more anti-pT1010 antibodies in the rabbit, and on Days 0, 26, 52, and 72 after immunization, the titers of sera were measured by ELISA. Afterward, antisera affinity purification and depletion were performed using a phosphopeptide column to remove non-specifically binding proteins, and a target antibody was concentrated, thereby isolating an antibody with relatively higher affinity for C-HRGVLS(pT)LIAGPV-amide from the isolated serum, compared to a non-phosphorylated peptide (C-HRGVLSTLIAGPV-amide), for further use. This is a process for preparing a phosphospecific antibody, and a process of removing a non-phosphospecific antibody binding to a non-phosphopeptide one more time using a non-phosphopeptide-attached column.

### Example 2. Confirmation of Antibody Reactivity

To confirm the reactivity of the antibody prepared in Example 1, the binding of a variant (TA) in which the threonine prototype at position 1010 in the amino acid sequence of NCAPG2 was substituted with Ala was confirmed by immunoprecipitation.

Specifically, 3×FLAG-NCAPG2 pT1010 wild-type (WT) and pT1010A mutant DNA were overexpressed in HEK293 cells using a transfection reagent (LTX/PLUS), and 24 hours later, cells were collected. After cell lysis, a total protein amount was more than 500 μg, and a reaction was performed at 4° C. and 20 rpm for more than 3 hours using FLAG M2 affinity beads to pull down NCAPG2. Subsequently, to increase purity (elute NCAPG2), an excessive amount of 3×Flag peptide was added, a competition process in which NCAPG2 binding to the FLAG beads was detached from the beads. It was performed using a centrifuge at 4° C. and 20 rpm for 30 minutes to 1 hour, and a supernatant was separated and then used.

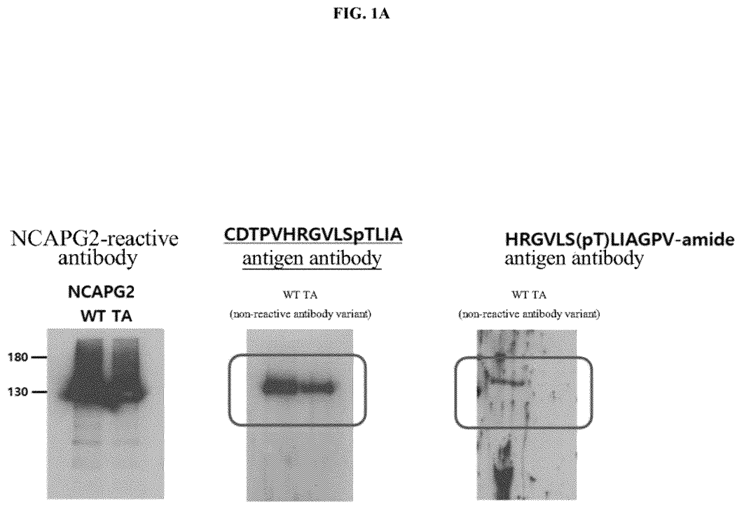

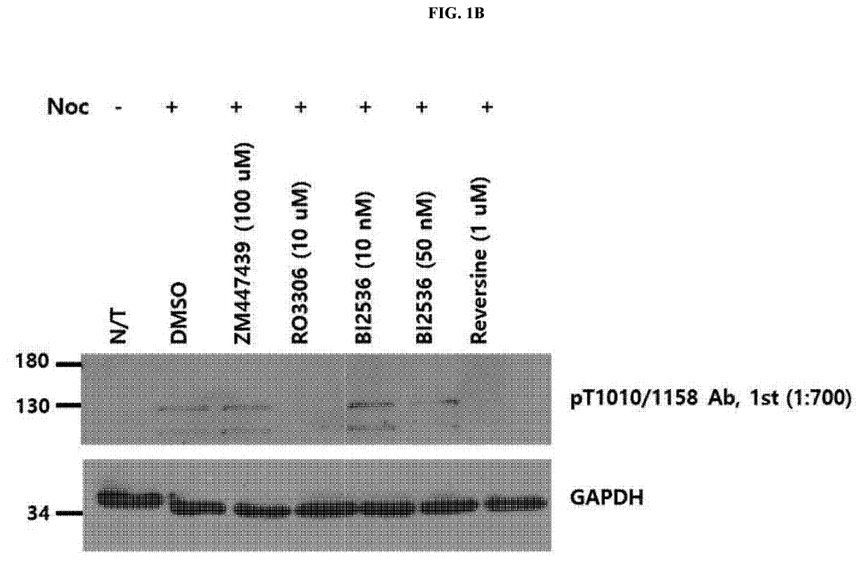

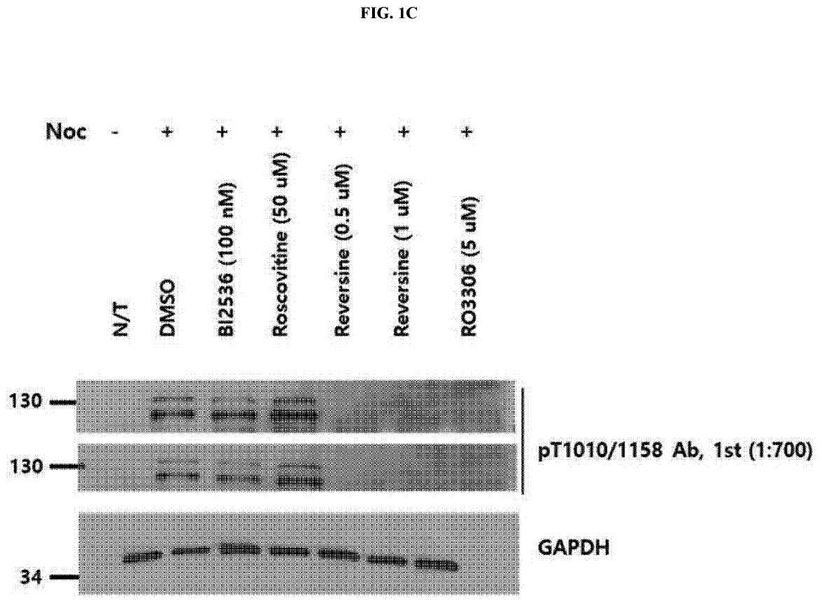

As a result, as shown in FIG. 1A, it was confirmed that the existing commercially-available NCAPG2 antibody (purchased from SIGMA ATLAS) was also detected in an NCAPG2 variant (TA) at an almost similar level, an antibody against a CDTPVHRGVLSpTLIA (SEQ ID NO: 3) antigen was reduced in a NCAPG2 variant (TA) but still detected. On the other hand, it was observed that the antibody against the C-HRGVLS(pT)LIAGPV-amide antigen, prepared in Example 1, was not detected in the NCAPG2 variant (TA), and it was confirmed that the NCAPG2 pT1010-specific antibody prepared in the present invention has reactivity specific to the phosphorylated part of threonine at position 1010 in the amino acid sequence of NCAPG2, and has much higher specificity to NCAPG2 pT1010, compared to the antibody against the CDTPVHRGVLSpTLIA antigen.

In addition, to confirm a phosphorylase inducing phosphorylation, after treating a breast cancer cell line MBAMB 231 with nocodazole, which is a mitosis-inducing reagent, and then with inhibitors of phosphorylases regulating mitosis for 2 hours, the reactivity of the NCAPG2 pT1010-specific antibody was confirmed. The phosphorylase inhibitors used herein are aurora kinase B: ZM447439, CDK1: RO3306, PLK1: BI2536, Mps1 and aurora kinase: reversine, and among these, as shown in FIG. 1B, it was confirmed that phosphospecific bands disappear after CDK1 and Mps1, and aurora kinase inhibition. It can be seen that this result was also shown even when the concentration conditions of the inhibitors had been changed, as shown in FIG. 1C.

### Example 3. Confirmation of Specificity of Antibody Using Immunocytochemistry

To confirm whether the NCAPG2 pT1010-specific antibody prepared in Example 1 can be used in cell staining, it was confirmed whether the reactivity of the antibody had sufficient specificity when reducing NCAPG2-specific expression by siRNA. To this end, 48 hours after treating MDA-MB-231 (program: X-013) with siGFP or siNCAPG2 by an AMAXA transfection method, staining was performed.

For staining, cells were fixed with 1% PFA and 0.2% Triton X-100, washed with PBS, blocked with 3% BSA/PBS, and then treated with a primary antibody at the following ratio.
1) anti NCAPG2 1:100/anti CenpC 1:5000/3% BSA/PBS
2) Old anti pT1010 1:200/anti CenpC 1:5000/3% BSA/PBS
3) New anti pT1010(1158, 2^{nd} purification) 1:200/anti CenpC 1:5000/3% BSA/PBS

Afterward, the resulting cells were washed with PBS, treated with a secondary antibody (Alexa 488 α-rabbit/Alexa 594 α-guinea pig) at 1:1000, mounted with VECTASHIELD, and then observed using a confocal microscope.

In addition, after knocking out NCAPG2 using a CRISPR-Cas9 system, an antibody was treated at the same ratio as above, and then the cells were stained in the same manner.

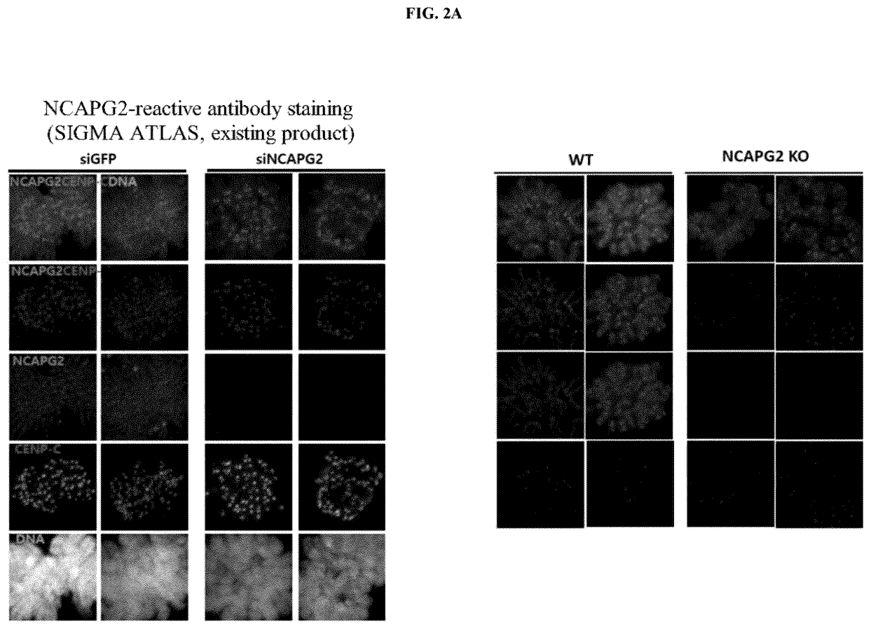

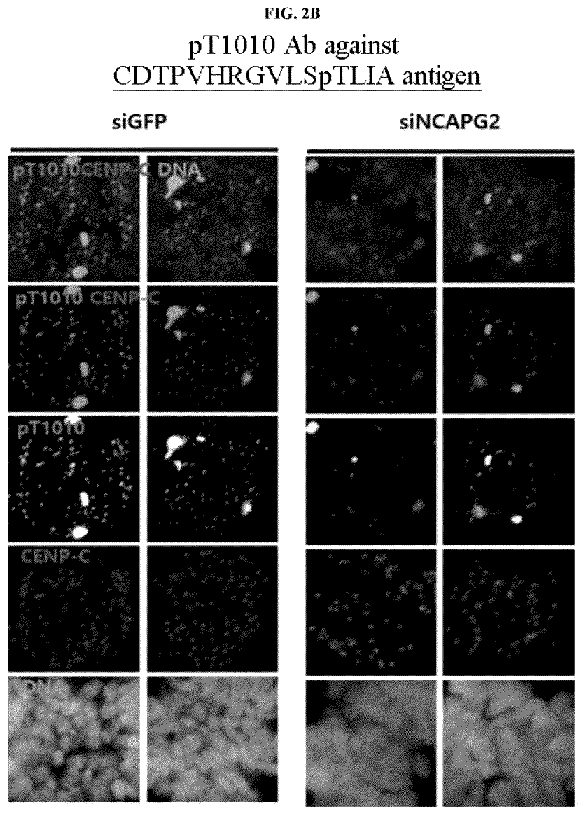

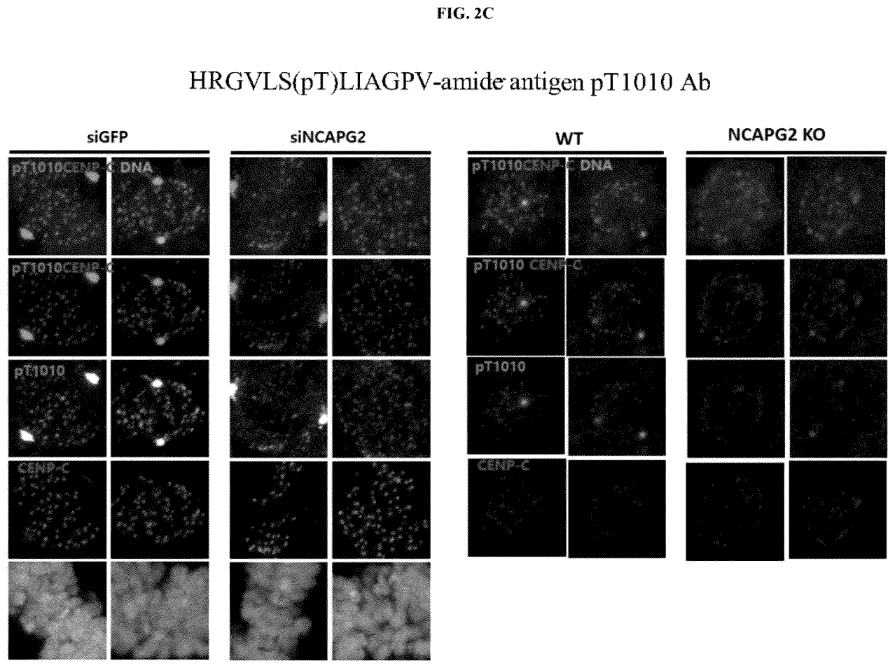

After performing immunocytochemistry in the above manner, the result for the existing commercially-available NCAPG2-reactive antibody is shown in FIG. 2A, the result for the anti-pT1010 antibody against the CDTPVHRGVLSpTLIA antigen is shown in FIG. 2B, the result for the anti-pT1010 antibody against the C-HRGVLS(pT)LIAGPV-amide antigen prepared in Example 1 is shown in FIG. 2C. By comparison of the locations of the parts stained with anti-CenpC and anti-pT1010 antibodies, it can be seen that when the NCAPG2 T1010 part is phosphorylated, the kinetochores in chromosomes are specifically stained.

When compared with FIG. 1A, it was observed that, compared to the previously prepared CDTPVHRGVLSpTLIA pT1010 antibody, the anti-pT1010 antibody against the HRGVLSpTLIAGPV-amide antigen is more clearly reduced in a TA variant, showing that, in terms of the selectivity or binding degree of an antibody, the anti-pT1010 antibody against the HRGVLSpTLIAGPV-amide antigen is more suitable. In addition, as compared with FIGS. 2A to 2C, the cell immunostaining experiment result shows a clearer staining pattern when using the HRGVLSpTLIAGPV-amide rabbit antibody, which had been boosted and purified one more time (2^{nd} purification). It was confirmed that when NCAPG2 expression was reduced using siRNA targeting mRNA or CRISPR-Cas9 targeting genomic DNA, kinetochores stained by the antibody of the present invention became weaker.

Accordingly, it can be seen that the selectivity or binding degree of the anti-pT1010 antibody against the HRGVLSpTLIAGPV-amide antigen according to the present invention is more suitable compared to the previously prepared CDTPVHRGVLSpTLIA pT1010 antibody.

### Example 4. Comparison of Immunostaining Patterns in Normal Cells and Cancer Cells

The phosphorylation of threonine at position 1010 of NCAPG2 is an important residue for interaction with PLK1 and normal cell division. Accordingly, to determine whether the phosphorylation pattern of threonine at position 1010 differs between normal cells and cancer cells, immunostaining experiments were performed using liver cancer (HepG2, SNU-449), breast cancer (MDA-MB-468), pancreatic cancer (KP-3, Panc1), liver cancer (A549, H1299), and prostate cancer (LNCaP) cell lines. As the antibody, a 1158 rabbit antibody (1158, new anti-pT1010) which had been boosted and purified one more time (2^{nd} purification) was used.

Each type of cancer cells was treated with 250 ng/ml of nocodazole 4 to 5 hours before harvesting. The cells were harvested using trypsin/EDTA, and then counted after washing with PBS. Afterward, the cells were re-suspended with PBS to reach 5×10⁵ cells/mL. In addition, the cells were put into a cuvette at 100 μL, and then spun down with Cytospin equipment at 1500 rpm for 5 minutes. For fixation, the cells were treated with a 1% paraformaldehyde solution with 0.25% Triton X-100 at room temperature for 10 minutes, and washed with PBS. Subsequently, after blocking (3% BSA/PBS), primary antibodies (anti-pT1010, 1:100/anti-CenpC, 1:5,000) were reacted overnight at 4° C. Next day, after washing with PBS, secondary antibodies (Alexa 488, anti-rabbit+Alexa 594, anti-guinea pig 1:1000) were reacted at room temperature for 2 hours, washed with PBS, and then stained with DAPI. In addition, after washing with PBS and mounting again, the cells were observed using a LSM780 confocal microscope.

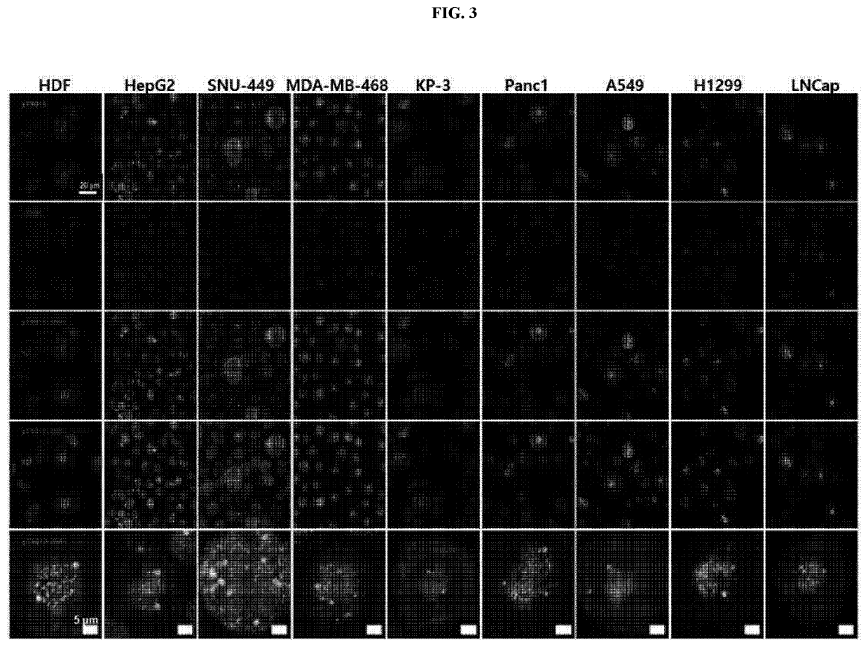

As a result, as shown in FIG. 3, it was confirmed that, in normal cells (HDF), when stained with the anti-pT1010 antibody, kinetochore component protein centromere protein C (CENP-C), which is binding sites of the chromosomes and spindles, is specifically stained during mitosis, whereas in cancer cells, there were many cases in which staining became intense, and kinetochore-specific staining did not occur in mitosis due to chromosomal instability.

### Example 5. Confirmation of Immunohistochemistry Result in Human Cancer Tissue

Immunohistochemistry is a method using an antigen-antibody reaction to specifically detect a target protein in tissue, and a method of visually confirming the location of a target protein reacting with an antigen through DAB staining to identify the target protein.

Using the immunohistochemical staining as above, NCAPG2 pT1010 expression was confirmed in non-tumor and tumor areas of human liver cancer, cholangiocarcinoma, and pancreatic cancer tissue.

### 5-1. Confirmation of Immunohistochemical Result in Liver Cancer Tissue

Immunohistochemistry was performed on a paraffin-embedded slide of human-derived liver cancer tissue prepared through a conventional tissue processing process through hydration, antigen retrieval using citrate buffer, blocking of endogenous peroxidase using hydrogen peroxide, blocking of a non-specific antibody reaction using normal horse serum, the antibody reaction specific to NCAPG2 pT1010 prepared in Example 1, and secondary antibody binding to a host of the NCAPG2 pT1010 antibody for detecting the antigen-antibody reaction. Afterward, 3 or 4 representative cross-sections of tumor and non-tumor areas in the tissue of each patient were photographed at 200× magnification, and the H-score system was utilized to quantify any positive reactions (the H-score is calculated by dividing the degree of positive reaction by 0, 1, 2, or 3, and then multiplying the proportions of cells corresponding to each value, and when all of the cells in the cross-section show intense positive reactions, a maximum value of 300 was assigned).

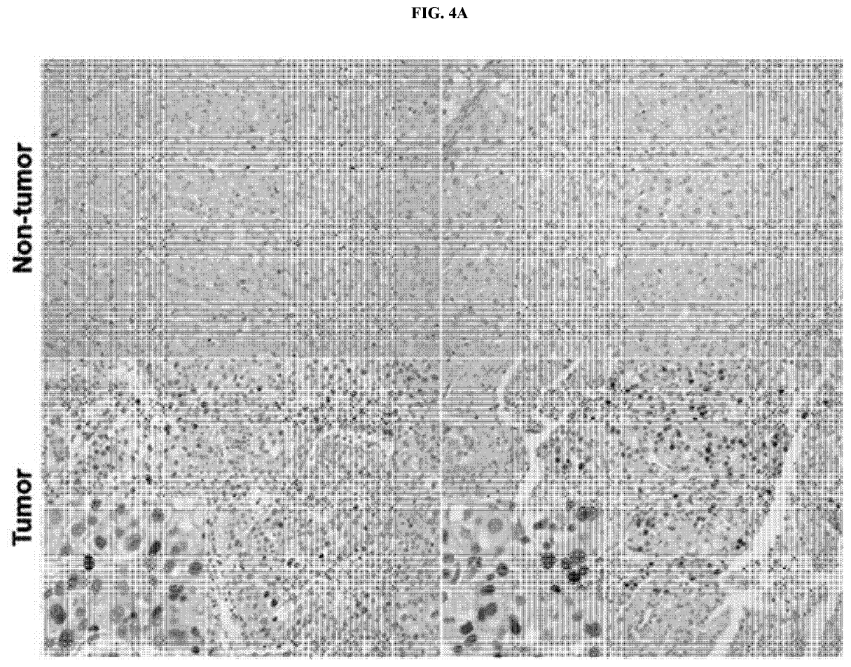

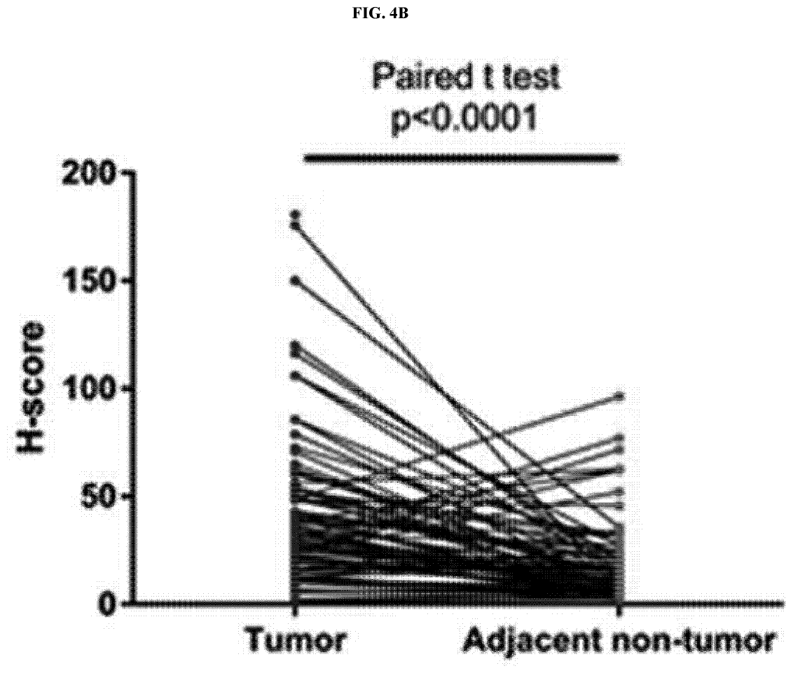

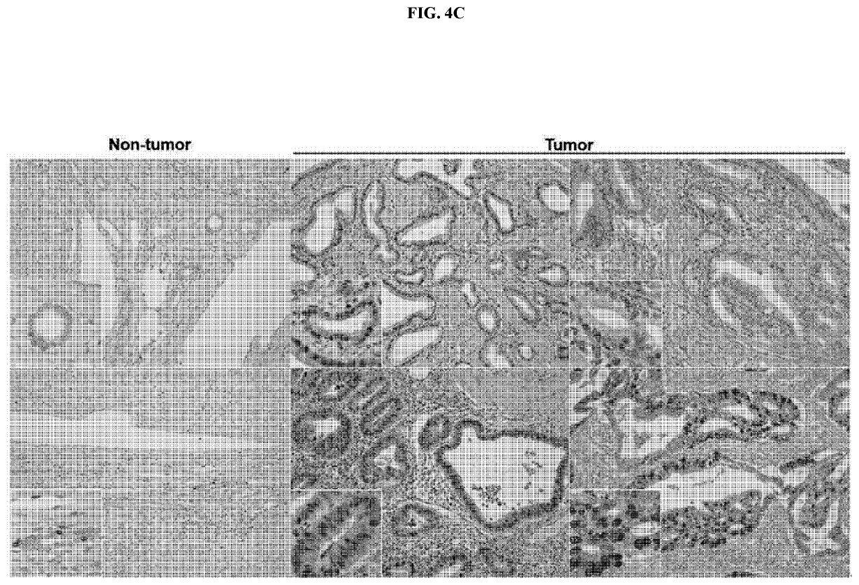

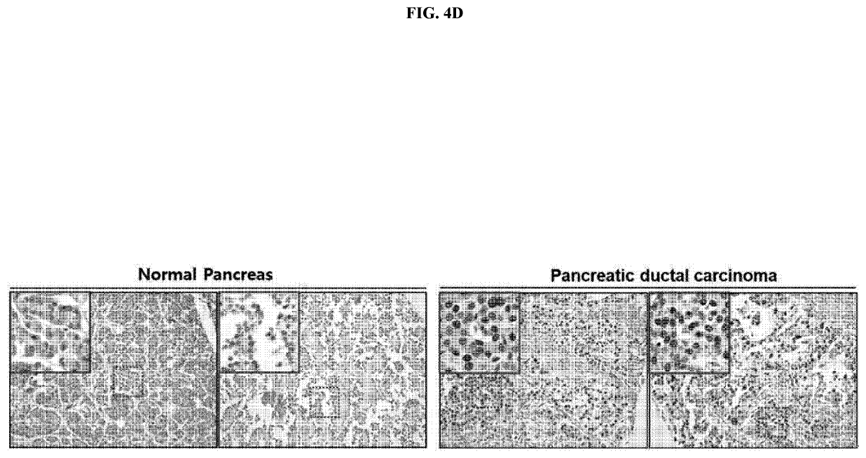

As a result of staining human liver cancer tissue by the above method and quantifying the result with respect to a positive reaction, as shown in FIGS. 4A and 4B, it was confirmed that as the anti-pT1010 antibody of NCAPG2 was stained at the location of hematoxylin used for nuclear and chromosomal staining in tissue staining, NCAPG2 pT1010 was expressed in most cell nuclei, and expression was significantly higher in the tumor areas compared to the non-tumor areas in the samples of 80 liver cancer patients.

### 5-2. Confirmation of Immunohistochemical Result in Cholangiocarcinoma Tissue

Immunohistochemistry for NCAPG2 pT1010, was performed on cholangiocarcinoma patient-derived tissue according to the method described in Example 4-1, and as a result, as shown in FIG. 4C, by confirming the staining location of the antibody against NCAPG2 pT1010, it was found that the expression of NCAPG2 pT1010 was very high in the nuclei of tumor cells in most cholangiocarcinoma tissue, and expression was significantly higher in the tumor areas compared to that of the non-tumor areas.

### 5-3. Confirmation of Immunohistochemical Result in Pancreatic Cancer Tissue

As a result of observing non-tumor/tumor areas in tissue samples derived from pancreatic cancer patients using a 200× microscope by the method described in Example 4-1, as shown in FIG. 4D, by confirming the staining location of the antibody against NCAPG2 pT1010, it was found that NCAPG2 pT1010 was specifically expressed in nuclei, and among these, expression was significantly high in pancreatic tissue compared to normal pancreatic tissue.

### Example 6. Confirmation of Expression Pattern of NCAPG2_pT1010 According to Degree of Differentiation of Pancreatic Cancer

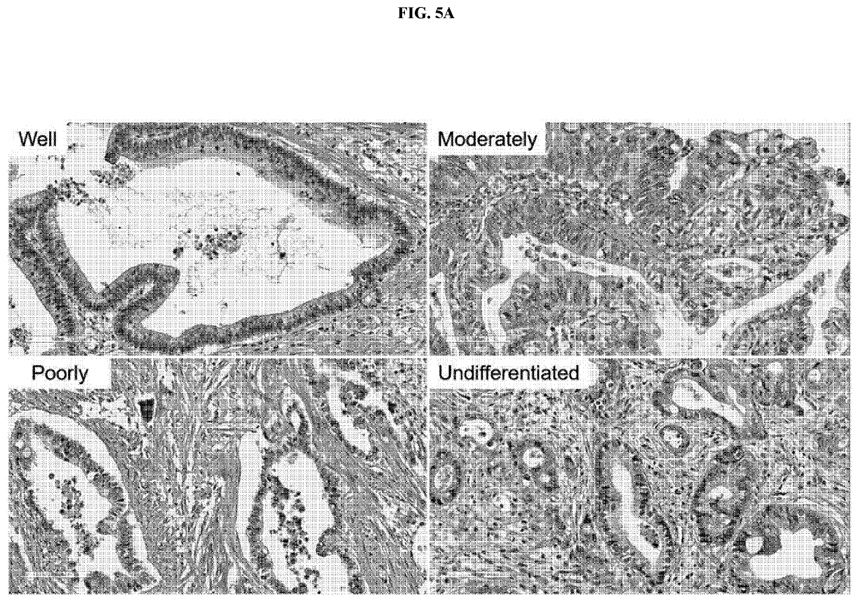

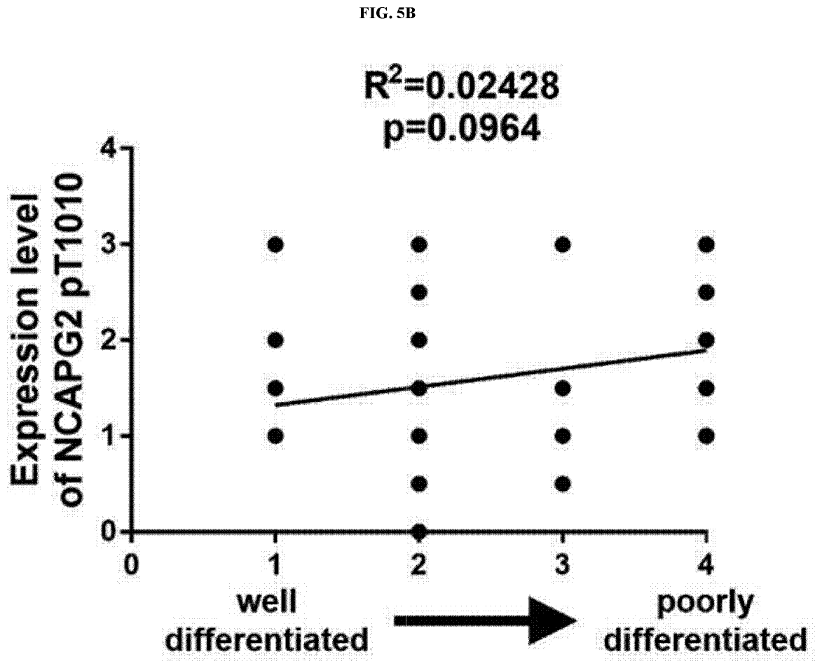

Immunohistochemistry for NCAPG2 pT1010, performed on a total of 126 cases of human pancreatic tissue, revealed that nucleus-specific staining occurred in the same way as confirmed in Example 5-3. The Q-score system was used to categorize the expression degree from 0 to 3, and a comparison was made between various types of clinical data, including an expression degree, the degree of tumor differentiation, a lymph node metastasis rate, the time to post-surgery recurrence, and the time to post-surgery death. As shown in FIGS. 5A and 5B, as the tumor was less differentiated, there was a tendency for the degree of NCAPG2 pT1010 expression in pancreatic cancer tissue to increase.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

### INDUSTRIAL APPLICABILITY

According to the present invention, an antibody for confirming whether threonine at position 1010 in the amino acid sequence of non-SMC condensin II complex subunit G2 (NCAPG2) is phosphorylated has high selectivity and high binding ability to NCAPG2 pT1010, inhibits the reactivity to NCAPG2 pT1010 in cancer cells treated with an inhibitor against phosphorylases for regulating mitosis, such as CDK1, PLK1, Mps1, and aurora kinase, and is detected at very high level in tumor tissue, compared to non-tumor tissue of a cancer patient, through immunohistochemical staining. Therefore, by confirming the phosphorylation of threonine at position 1010 of NCAPG2 using the antibody according to the present invention, it is expected to be useful for cancer-related research and development fields, including cancer diagnosis or screening of an anticancer drug candidate. Therefore, the antibody according to the present invention has industrial applicability.

1. An antibody for confirming whether threonine at position 1010 from the N terminus of the amino acid sequence of non-SMC condensin II complex subunit G2 (NCAPG2) is phosphorylated,
wherein the antibody recognizes a peptide represented by the amino acid sequence of SEQ ID NO: 1 as an antigen, and specifically binds to threonine at position 1010 from the N terminus of the amino acid sequence of the phosphorylated NCAPG2.

2. The antibody of claim 1, wherein the amino acid sequence of NCAPG2 is represented by SEQ ID NO: 2.

3. (canceled)

4. A method of providing information for cancer diagnosis, comprising:
confirming whether threonine at position 1010 from the N terminus of the amino acid sequence of non-SMC condensin II complex subunit G2 (NCAPG2) is phosphorylated by reacting NCAPG2-expressing cells with the antibody of claim 1.

5. The method of claim 4, further comprising:
diagnosing cancer when threonine at position 1010 from the N terminus of the amino acid sequence of NCAPG2 is phosphorylated.

6. The method of claim 4, wherein the higher the degree of phosphorylation of the threonine at position 1010 from the N terminus of the amino acid sequence of NCAPG2, the lower the degree of differentiation of tumor cells.

7. A method of screening a candidate for an anticancer drug, comprising:
(a) treating cancer cells with a candidate;
(b) confirming whether threonine at position 1010 from the N terminus of the amino acid sequence of non-SMC condensin II complex subunit G2 (NCAPG2) is phosphorylated by reacting the cancer cells treated with the candidate with the antibody of claim 1; and
(c) selecting the candidate as an anticancer drug candidate when the phosphorylation of threonine at position 1010 from the N terminus of the amino acid sequence of NCAPG2 in (b) is inhibited.

8. The method of claim 7, wherein the anticancer drug candidate comprises one or more selected from the group consisting of a polo-like kinase 1 (PLK1) inhibitor, a monopolar spindle 1 (Mps1) inhibitor, an aurora kinase inhibitor, and a cyclin-dependent kinase 1 (CDK1) inhibitor.

9. The method of claim 8, wherein the PLK1 inhibitor, the Mps1 inhibitor, the aurora kinase inhibitor, or the CDK1 inhibitor is one or more selected from the group consisting of a nucleotide, DNA, RNA, an amino acid, an aptamer, a protein, a compound, a natural substance, and a natural extract.

10-16. (canceled)
